(19) United States
(12) Patent Application Publication
(10) Pub. No.: US 2025/0339360 A1
(43) Pub. Date: Nov. 6, 2025

(54) COSMETIC COMPOSITIONS COMPRISING RETINOIDS AND MOISTURIZERS AND METHODS FOR STABLIZING RETINOIDS

(71) Applicant: ELC Management LLC, Melville, NY (US)

(72) Inventors: Tracey Anne MCGRORY, Northport, NY (US); Peter John TSOLIS, Huntington Station, NY (US); Sydney Fiona SCHAEFER, Long Island City, NY (US); Jennifer Marie SIEGEL, Farmingdale, NY (US)

(73) Assignee: ELC Management LLC, Melville, NY (US)

(21) Appl. No.: 19/188,408

(22) Filed: Apr. 24, 2025

Related U.S. Application Data
(60) Provisional application No. 63/641,227, filed on May 1, 2024.

(51) Int. Cl. A61K 8/67; A61K 8/02; A61K 8/37; A61K 8/81; A61K 8/92; A61Q 19/08

(52) U.S. Cl. CPC A61K 8/671; A61K 8/0216; A61K 8/375; A61K 8/8111; A61K 8/927; A61Q 19/08; A61K 2800/52; A61K 2800/5922

(57) ABSTRACT

Cosmetic compositions having retinoid stability may comprise about 70 wt % to about 85 wt % of at least one emollient, based on total mass, and up to about 0.3 wt % of at least one retinoid, based on total mass. Suitable emollients may include, for example, at least glyceryl triacetyl ricinoleate, bis-diglyceryl polyacyladipate-2, hydrogenated polyisobutene, synthetic beeswax, and diisostearyl malate. The at least one retinoid may comprise hydroxypinacolone retinoate.

### FIELD

The present disclosure generally relates to cosmetic compositions, and more specifically, to cosmetic compositions containing stabilized retinoids.

### BACKGROUND

Retinoids are frequently used in the personal care industry due to their biological benefits and efficacies. For example, retinol is a regulator of epidermal cell growth, normal cell differentiation, and cell maintenance. However, retinoids are also recognized for their instability under a range of degradation conditions. Upon exposure to UV light, retinoids are subject to photoreactions, such as photoisomerization, photopolymerization, photooxidation, and photodegradation. Retinoids are also sensitive to oxidation and heat, thereby undergoing degradation when exposed to oxygen and elevated temperatures. In most cases, retinoid degradation results in a loss of biological efficacy.

The personal care industry has attempted to rectify retinoid stability issues by employing technologies such as encapsulating retinoids, making nanoformulations, creating new retinoid derivatives, and so on. For example, retinol ester derivatives like retinyl palmitate, retinyl propionate, retinyl acetate, and retinyl retinoate have been created and used in personal care products with some degree of success. This approach is complicated, and it may be difficult to predict whether a stability increase will occur. Moreover, each new retinoid derivative may have its own efficacy and stability profile, which may require further dedicated research efforts to optimize its use in personal care products. Increased development and consumer costs may result from these difficulties.

### SUMMARY

In some embodiments, cosmetic compositions of the present disclosure comprise: about 70 wt % to about 85 wt % of at least one emollient, based on total mass, and up to about 0.3 wt % of at least one retinoid, based on total mass.

In some or other embodiments, cosmetic compositions of the present disclosure comprise: about 70 wt % to about 85 wt % of at least one emollient, based on total mass, the at least one emollient comprising at least glyceryl triacetyl ricinoleate, bis-diglyceryl polyacyladipate-2, hydrogenated polyisobutene, synthetic beeswax, and diisostearyl malate; about 0.001 wt % to about 0.3 wt % of at least one retinoid, based on total mass, the at least one retinoid comprising at least hydroxypinacolone retinoate; about 15 wt % to about 25 wt % of at least one occlusive, based on total mass, the at least one occlusive comprising at least Helianthus annuus (sunflower) seed wax, Oryza sativa (rice) bran wax, squalane, and tocopherol; about 0.5 wt % to about 2 wt % of at least one viscosity-decreasing agent, based on total mass, the at least one viscosity-decreasing agent comprising at least dimethyl isosorbide; and about 0.005 wt % to about 3 wt % of at least one humectant, based on total mass, the at least one humectant comprising at least caprylyl glycol.

### BRIEF DESCRIPTION OF THE DRAWINGS

Not applicable.

### DETAILED DESCRIPTION

The present disclosure generally relates to cosmetic compositions, and more specifically, to cosmetic compositions containing stabilized retinoids.

As discussed above, retinoid stability may be problematic in cosmetic compositions and other personal care products. Conventional approaches for stabilizing retinoids may involve development of complicated formulations and even synthesis and testing of new retinoid derivatives. These difficulties may result in increased costs.

The present disclosure provides compositions and methods that may meet the foregoing needs and provide related advantages as well. In particular, the present disclosure demonstrates that cosmetic compositions containing at least one emollient and at least one retinoid may surprisingly maintain retinoid stability over extended post-formulation (storage) times. For example, cosmetic compositions of the present disclosure may maintain a retinoid concentration within about 10% of an as-formulated concentration value over at least about 6 months. Surprisingly, the compositions may maintain retinoid stability even when stored under heating conditions (40° C.) and in the presence of moisture (60% relative humidity). The surprising storage stability of the presently described cosmetic compositions may afford extended shelf life and a potentially more active product in the hands of the consumer. Moreover, because one does not have to account for storage degradation and its impact on efficacy, it may be possible to formulate cosmetic compositions according to the present disclosure while using less retinoid than would otherwise be possible. Decreased costs may be realized as a result.

The cosmetic compositions of the present disclosure may be produced in a variety of forms. Suitable forms may include, for example, solids, liquids, serums, gels, creams, or the like. Solid forms may be particularly desirable. Suitable solid forms may be malleable or non-malleable. In such forms, the cosmetic compositions may be aqueous-based (contain an aqueous fluid) or non-aqueous-based (lack added water). It may be particularly desirable for solid forms of the cosmetic compositions to lack added water and be substantially anhydrous.

Retinoids are vitamin A derivatives and may be natural or synthetic. Nonlimiting examples of suitable retinoids may include, but are not limited to, retinol, retinal, retinoic acid, isotretinoin, alitretinoin, etretinate, acitretin, adapalene, bexarotene, tazarotene, trifarotene, retinyl esters, retinoic acid esters, and the like. Suitable retinyl esters may include, for example, retinyl palmitate, retinyl propionate, retinyl acetate, the like, or any combination thereof. Suitable esters of retinoic acid may include, but are not limited to, hydroxypinacolone retinoate, retinyl retinoate, the like, or any combination thereof. Retinyl retinoate may be classified as both a retinyl ester and an ester of retinoic acid.

In some examples, the at least one retinoid may comprise a retinyl ester, a retinoic acid ester, or a mixture thereof. In still more specific examples, the at least one retinoid may comprise at least hydroxypinacolone retinoate.

The cosmetic compositions of the present disclosure may comprise one retinoid or more than one retinoid, including any of the foregoing retinoids singularly or in any combination. One, two, three, four, five, or even more retinoids may be present in the cosmetic compositions described herein. When multiple retinoids are present, the multiple retinoids may be present in any proportion with respect to one another.

In the cosmetic compositions described herein, the at least one retinoid may be present over a range of concentrations. In non-limiting examples, the concentration of the at least one retinoid in the cosmetic compositions may range from about a1 wt % to about a2 wt %, wherein a1 and a2 are, independently, 0.0001, 0.0005, 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 21, 22, 23, 24, or 25, based on total mass of the cosmetic composition and where a1<a2. For example, the at least one retinoid may be present in the cosmetic compositions in an amount ranging from about 0.0001 wt % to about 25 wt %, or about 0.001 wt % to about 15 wt %, or about 0.01 wt % to about 5 wt %, or about 0.05 wt % to about 0.5 wt %, or about 0.01 wt % to about 1 wt %, or about 0.001 wt % to about 0.3 wt %, or about 0.01 wt % to about 0.3 wt %, or about 0.01 wt % to about 0.1 wt %, or about 0.05 wt % to about 0.1 wt %, based on total mass of the cosmetic composition. Hydroxypinacolone retinoate may be present in any of the foregoing amounts.

The at least one retinoid may be dispersed (admixed) within at least one moisturizer in the cosmetic compositions disclosed herein. The dispersion of the at least one retinoid within the at least one moisturizer may be homogeneous or heterogeneous. Dispersion may be achieved by blending the at least one retinoid and the at least one moisturizer together in a suitable manner, such as by agitating a mixture of the at least one retinoid and the at least one moisturizer. Without being bound by theory or mechanism, the at least one moisturizer is believed to define a matrix that at least partially surrounds the at least one retinoid and conveys stabilization thereto.

Moisturizers moisten the skin and are common ingredients in the personal care industry. Due to their function of skin moistening, moisturizers may bring many skin benefits such as, for example, anti-inflammation properties, antipruritic properties, antimitotic properties, wound healing properties, and so on. Based on how moisturizers aid in moistening the skin, moisturizers may be categorized into three main categories: humectants, emollients, and occlusives. These categories are not exclusive to each other. For example, a given moisturizer may help in moistening the skin in multiple ways and may belong to more than one moisturizer category. Examples of suitable humectants, emollients, and occlusives, as well as suitable amounts thereof, are discussed further below.

The total amount of moisturizers in the cosmetic compositions disclosed herein may range from about b1 wt % to about b2 wt %, wherein b1 and b2 are, independently, 80, 81, 82, 83, 84, 85, 86, 87, 88, 89, 90, 91, 92, 93, 94, 95, 96, 97, 98, 99, 99.5, 99.9, 99.999, or 99.9999, based on total mass of the cosmetic composition and where b1<b2. For example, the total amount of moisturizers in the cosmetic compositions may range from about 80 wt % to about 99.999 wt %, or about 90 wt % to about 99.99 wt %, or about 95 wt % to about 99.9 wt %, based on total mass of the cosmetic composition.

The cosmetic compositions and the at least one moisturizer may contain at least one humectant. Humectants are hydrophilic compounds that may aid in attracting water to the skin. If used, humectants may be present in the cosmetic compositions in an amount ranging from about c1 wt % to about c2 wt %, where c1 and c2 are, independently, 0.001, 0.005, 0.01, 0.05, 0.1, 0.5, 1, 1.5, 2, 2.5, 3, 3.5, 4, 4.5, or 5, based on total mass of the cosmetic composition and where c1<c2. For example, an amount of the at least one humectant, if present, may range from about 0.001 wt % to about 5 wt %, or about 0.005 wt % to about 3 wt %, or about 0.1 wt % to about 1 wt %, based on total mass of the cosmetic composition.

Nonlimiting examples of suitable humectants may include, for example, glycols, glycol ethers, sugars, and the like. Suitable glycols may be in monomeric or polymeric form and include polyethylene glycols and polypropylene glycols such as PEG 4-200. The number of repeating ethylene oxide or propylene oxide units in suitable polyethylene glycols and/or polypropylene glycols may range from about 4 to about 200 repeating ethylene oxide or propylene oxide units. Other C₂₋₁₀ alkylene glycols suitable for use herein may include, for example, butylene glycol, pentylene glycol, caprylyl glycol, and the like. Sugars, some of which are also polyhydric alcohols, may also be suitable humectants. Examples of suitable sugars (or sugar sources) for use herein may include, for instance, glucose, fructose, honey, hydrogenated honey, inositol, maltose, mannitol, maltitol, sorbitol, sucrose, xylitol, xylose, hyaluronic acid, hyaluronic acid salts like sodium hyaluronate, and the like. U rea may also be a suitable humectant in some cases.

In more specific examples, if a humectant is present in the cosmetic compositions, the humectant may comprise one or more of a hyaluronic acid salt, a C₂₋₁₀ alkylene glycol, or any combination thereof. In still more specific examples, the humectant, if present, may comprise sodium hyaluronate, a C₆₋₁₀ alkylene glycol, or any mixture thereof, which may be present in any mass ratio with respect to one another. Caprylyl glycol is an illustrative C₆₋₁₀ alkylene glycol that may be suitable for use in the cosmetic compositions disclosed herein. For example, the cosmetic compositions may comprise about 0.05 wt % to about 0.8 wt % caprylyl glycol, based on total mass of the cosmetic composition.

The cosmetic compositions and the at least one moisturizer may contain at least one occlusive. Occlusives are compounds that may form a barrier layer to aid in retaining moisture in the skin. If used, occlusives may be present in the cosmetic compositions in an amount ranging from about d1 wt % to about d2 wt %, where d1 and d2 are, independently, 5, 6, 7, 8, 9, 10, 11, 12, 13, 14, 15, 16, 17, 18, 19, 20, 21, 22, 23, 24, or 25, based on total mass of the cosmetic composition and where d1<d2. For example, an amount of the at least one occlusive, if present, may range from about 5 wt % to about 25 wt %, or about 8 wt % to about 20 wt %, or about 12 wt % to about 18 wt %, or about 15 wt % to about 25 wt %, based on total mass of the cosmetic composition.

Nonlimiting examples of suitable occlusives may include, but are not limited to, dimethicone, dimethiconol, Helianthus annuus (sunflower) seed wax, mineral oil, Oryza sativa (rice) bran wax, petrolatum, squalane, tocopherol, or any combination thereof. In some examples, the at least one occlusive may be present and include one or more of Helianthus annuus (sunflower) seed wax, Oryza sativa (rice) bran wax, squalane, tocopherol, or any combination thereof. In some examples, the at least one occlusive may include each of Helianthus annuus (sunflower) seed wax, Oryza sativa (rice) bran wax, squalane, and tocopherol, which may be present in any mass ratio with respect to one another. For example, suitable amounts of Helianthus annuus (sunflower) seed wax may range from about 1 wt % to about 10 wt %, suitable amounts of Oryza sativa (rice) bran wax may range from about 1 wt % to about 8 wt %, suitable amounts of squalane may range from about 1 wt % to about 15 wt %, and suitable amounts of tocopherol may range from about 0.0001 wt % to about 0.001 wt %, each based on total mass of the cosmetic composition.

The cosmetic compositions and the at least one moisturizer may contain at least one emollient. Emollients are compounds that may aid in hydrating dry, flaky skin. Emollients may be present in the cosmetic compositions in an amount ranging from about e1 wt % to about e2 wt %, where e1 and e2 are, independently, 40, 45, 50, 55, 60, 65, 70, 75, 80, or 85, based on total mass of the cosmetic composition and where e1<e2. For example, an amount of the at least one emollient may range from about 40 wt % to about 85 wt %, or about 40 wt % to about 80 wt %, or about 50 wt % to about 70 wt %, or about 70 wt % to about 85 wt %, or about 60 wt % to about 65 wt %, based on total mass of the cosmetic composition.

Individual emollients may be present in the cosmetic compositions in an amount of about 40 wt % or below. In non-limiting examples, individual emollients may be present at about 0.01 wt % or greater, or about 0.05 wt % or greater, or about 0.1 wt % or greater, or about 0.5 wt % or greater, or about 1 wt % or greater, or about 2 wt % or greater, or about 5 wt % or greater, or about 10 wt % or greater, or about 20 wt % or greater, based on total mass of the cosmetic composition, and provided that the total emollients present are within the range of about e1 wt % to about e2 wt %, recited above, and individual emollients are present in an amount of about 40 wt % or below.

Nonlimiting examples of suitable emollients may include, but are not limited to, acetylated hydrogenated cottonseed glyceride, acetylated hydrogenated tallow glyceride, acetylated hydrogenated tallow glycerides, acetylated lanolin, acetylated lanolin alcohol, acetylated palm kernel glycerides, amino bispropyl dimethicone, apricot kernel oil PEG-6 esters, behenamidopropyl dimethylamine, behenyl/isostearyl beeswax, bis-diglyceryl polyacyladipate-2, Butyrospermum parkii (shea) butter, C10-C30 cholesterol/lanosterol esters, C10-C18 triglycerides, C12-C15 alkylbenzoate, canola oil glyceride, caprylic/capric/diglyceryl succinate, caprylic/capric glycerides, caprylic/capric triglyceride PEG-4 esters, cetearyl octanoate, cetearyl stearate, cetyl dimethicone copolyol, cholesterol, cholesteryl stearate, cocoglycerides, coconut alcohol, corn glycerides, corn oil PEG-6 esters, dicocodimethylamine dilinoleate, diethylaminoethyl PEG-5 laurate, diethylaminoethyl stearate, dihydrogenated tallow methylamine, dihydrolanosterol, diisostearyl malate, dimethoxysilyl ethylenediaminopropyl dimethicone, dioctyldodecyl stearoyl glutamate, dipentaerythrityl hexacaprylate/hexacaprate, dipentaerythrityl hexahydroxystearate, dipentaerythrityl hexahydroxystearate/isostearate, dipentaerythrityl hexahydroxystearate/stearate/rosinate, dipentaerythrityl pentaoctanoate/behenate, diphenyl dimethicone, dthylene dihydrogenated tallowamide, fluoro C2-C8 alkyldimethicone, glycereth-20 stearate, glycereth-25 PCA isostearate, glycereth-26 phosphate, glycereth-5 lactate, glycereth-7 benzoate, glycereth-7 diisononanoate, glycereth-8 hydroxystearate, glyceryl/sorbitol oleate/hydroxystearate, glyceryl arachidate, glyceryl arachidonate, glyceryl behenate, glyceryl caprylate, glyceryl caprylate/caprate, glyceryl citrate/lactate/linoleate/oleate, glyceryl cocoate, glyceryl diisostearate, glyceryl dilaurate, glyceryl dipalmitate, glyceryl distearate, glyceryl erucate, glyceryl glycyrrhetinate, glyceryl hydroxystearate, glyceryl isopalmitate, glyceryl isostearate, glyceryl isotridecanoate/stearate/adipate, glyceryl lanolate, glyceryl laurate, glyceryl linoleate, glyceryl myristate, glyceryl oleate, glycerylpalmitoleate, glyceryl sesquioleate, glyceryl stearate, glyceryl stearate diacetate, glyceryl stearate lactate, glyceryl triacetyl ricinoleate, glycol octanoate, glycol oleate, glycol palmitate, glycol ricinoleate, glycol stearate, hexyldeceth-20, hydrogenated castor oil, hydrogenated palm oil, hydrogenated peanut oil, hydrogenated polyisobutene, hydrogenated soybean glycerides, hydrogenated vegetable glycerides, hydroxycetyl isostearate, hydroxylated lanolin, isopropyl lanolate, isopropyl palmitate, isosteareth-10 stearate, isostearic/myristic glycerides, isostearic acid, isostearoyl isostearyl stearate, jojoba esters, lanolin alcohol, laurylmethicone copolyol, lecithin, linseed acid, lysolecithin, Mangifera indica (mango) seed butter, methyl gluceth-10, methyl glucose isostearate, methyl glucose laurate, methyl glucose sesquicaprylate/sesquicaprate, methyl glucose sesquicocoate, methyl glucose sesquiisostearate, methyl glucose sesquilaurate, methyl glucose sesquioleate, methyl glucose sesquistearate, methyl isostearate, methylsilanol PEG-7 glyceryl cocoate, neopentyl glycol diisostearate, neopentyl glycol dilaurate, octoxyglyceryl behenate, octoxyglyceryl palmitate, octyl linoleoyl stearate, octyldecyl oleate, oleyl alcohol, olive oil PEG-10 esters, palm glyceride, palm glycerides, palm kernel alcohol, palm kernel glycerides, peanut glycerides, PEG-10 castor oil, PEG-10 cocoate, PEG-10 dioleate, PEG-10 glyceryl oleate, PEG-10 glyceryl stearate, PEG-10 hydrogenated lanolin, PEG-10 hydrogenated tallow amine, PEG-10 isolauryl thioether, PEG-10 isostearate, PEG-10 lanolate, PEG-10 lanolin, PEG-10 laurate, PEG-10 oleate, PEG-10 olive glycerides, PEG-10 sorbitan laurate, PEG-10 soya sterol, PEG-10 soyamine, PEG-10 stearamine, PEG-10 stearate, PEG-10 tallate, PEG-10 tallow aminopropylamine, PEG-100 castor oil, PEG-100 hydrogenated castor oil, PEG-100 lanolin, PEG-11 avocado glycerides, PEG-11 babassu glycerides, PEG-11 castor oil, PEG-11 cocamide, PEG-11 oleate, PEG-11 tallow amine, PEG-12 beeswax, PEG-12 dilaurate, PEG-12 dioleate, PEG-12 distearate, PEG-12 ditallate, PEG-12 glyceryl dioleate, PEG-12 glyceryl laurate, PEG-12 isostearate, PEG-12 laurate, PEG-12 oleate, PEG-12 palm kernel glycerides, PEG-12 stearate, PEG-12 tallate, PEG-120 distearate, PEG-120 jojoba acid, PEG-120 jojoba alcohol, PEG-120 methyl glucose dioleate, PEG-120 propylene glycol stearate, PEG-13 diphenylol propane, PEG-13 hydrogenated tallow amide, PEG-13 mink glycerides, PEG-13 octanoate, PEG-14 avocado glycerides, PEG-14 laurate, PEG-14 oleate, PEG-14 stearate, PEG-14 tallate, PEG-140 glyceryl tristearate, PEG-15 butanediol, PEG-15 castor oil, PEG-15 cocamine, PEG-15 cocamine oleate/phosphate, PEG-15 cocoate, PEG-15 cocomonium chloride, PEG-15 cocopolyamine, PEG-15 glyceryl isostearate, PEG-15 glyceryl laurate, PEG-15 glyceryl oleate, PEG-15 glyceryl ricinoleate, PEG-15 hydrogenated tallow amine, PEG-15 hydroxystearate, PEG-15 jojoba acid, PEG-15 jojoba alcohol, PEG-15 lanolate, PEG-15 oleamine, PEG-15 oleate, PEG-15 soyamine, PEG-15 stearamine, PEG-15 tallow aminopropylamine, PEG-15 tallow polyamine, PEG-150 dilaurate, PEG-150 dioleate, PEG-150 distearate, PEG-150 lanolin, PEG-150 pentaerythrityl tetrastearate, PEG-16 hydrogenated castor oil, PEG-16 oleate, PEG-16 soya sterol, PEG-16 tallate, PEG-175 distearate, PEG-18 castor oil dioleate, PEG-18 glyceryl oleate/cocoate, PEG-18 palmitate, PEG-18 stearate, PEG-2 castor oil, PEG-2 cocamine, PEG-2 cocomonium chloride, PEG-2 diisononanoate, PEG-2 dilaurate, PEG-2 dioctanoate, PEG-2 distearate, PEG-2 hydrogenated castor oil, PEG-2 hydrogenated tallow amine, PEG-2 laurate, PEG-2 laurate SE, PEG-2 oleamide, PEG-2 oleamine, PEG-2 oleammonium chloride, PEG-2 oleate, PEG-2 oleate SE, PEG-2 ricinoleate, PEG-2 sorbitan isostearate, PEG-2 soyamine, PEG-2 stearamine, PEG-2 stearate, PEG-2 stearate SE, PEG-2 stearmonium chloride, PEG-20 almond glycerides, PEG-20 beeswax, PEG-20 castor oil, PEG-20 cocamide, PEG-20 cocamine, PEG-20 corn glycerides, PEG-20 dilaurate, PEG-20 dioleate, PEG-20 distearate, PEG-20 evening primrose glycerides, PEG-20 glyceryl laurate, PEG-20 glyceryl oleate, PEG-20 glyceryl ricinoleate, PEG-20 glyceryl stearate, PEG-20 hydrogenated castor oil, PEG-20 hydrogenated lanolin, PEG-20 hydrogenated palm oil glycerides, PEG-20 hydrogenated tallow amine, PEG-20 lanolate, PEG-20 lanolin, PEG-20 laurate, PEG-20 mannitan laurate, PEG-20 methyl glucose distearate, PEG-20 methyl glucose sesquicaprylate/sesquicaprate, PEG-20 methyl glucose sesquilaurate, PEG-20 methyl glucose sesquistearate, PEG-20 myristate, PEG-20 oleate, PEG-20 palmitate, PEG-20 sorbitan beeswax, PEG-20 sorbitan cocoate, PEG-20 sorbitan isostearate, PEG-20 stearate, PEG-20 tallate, PEG-20 tallowate, PEG-20-PPG-10 glyceryl stearate, PEG-200 castor oil, PEG-200 glyceryl tallowate, PEG-200 hydrogenated castor oil, PEG-200 montanate, PEG-200 trihydroxystearin, PEG-22/dodecyl glycol copolymer, PEG-23 glyceryl laurate, PEG-23 oleate, PEG-23 stearate, PEG-24 hydrogenated lanolin, PEG-24 lanolin, PEG-25 castor oil, PEG-25 glyceryl oleate, PEG-25 glyceryl stearate, PEG-25 glyceryl trioleate, PEG-25 hydrogenated castor oil, PEG-25 phytosterol, PEG-25 propylene glycol stearate, PEG-25 soya sterol, PEG-25 stearate, PEG-26 castor oil, PEG-26 jojoba acid, PEG-26 jojoba alcohol, PEG-27 lanolin, PEG-28 glyceryl tallowate, PEG-29 castor oil, PEG-3/PPG-2 glyceryl/sorbitol hydroxystearate/isostearate, PEG-3 castor oil, PEG-3 cocamide, PEG-3 cocamine, PEG-3 dipalmitate, PEG-3 distearate, PEG-3 lanolate, PEG-3 lauramide, PEG-3 oleamide, PEG-3 oleate, PEG-3 sorbitan oleate, PEG-3 sorbitan stearate, PEG-3 tallow aminopropylamine, PEG-30 castor oil, PEG-30 dipolyhydroxystearate, PEG-30 glyceryl cocoate, PEG-30 glyceryl laurate, PEG-30 glyceryl oleate, PEG-30 glyceryl stearate, PEG-30 hydrogenated castor oil, PEG-30 hydrogenated lanolin, PEG-30 hydrogenated tallow amine, PEG-30 lanolin, PEG-30 oleamine, PEG-30 sorbitan tetraoleate, PEG-30 sorbitol tetraoleate laurate, PEG-30 soya sterol, PEG-30 stearate, PEG-32 dilaurate, PEG-32 dioleate, PEG-32 distearate, PEG-32 oleate, PEG-32 stearate, PEG-33 castor oil, PEG-35 almond glycerides, PEG-35 castor oil, PEG-35 hydrogenated castor oil, PEG-35 lanolin, PEG-35 stearate, PEG-36 castor oil, PEG-36 oleate, PEG-36 stearate, PEG-4 castor oil, PEG-4 diheptanoate, PEG-4 dilaurate, PEG-4 dioleate, PEG-4 distearate, PEG-4 isostearate, PEG-4 lanolate, PEG-4 laurate, PEG-4 octanoate, PEG-4 oleamide, PEG-4 oleate, PEG-4 polyglyceryl-2 stearate, PEG-4 stearate, PEG-4 tallate, PEG-40 castor oil, PEG-40 glyceryl cocoate, PEG-40 hydrogenated castor oil, PEG-40 hydrogenated castor oil PCA isostearate, PEG-40 hydrogenated tallow amine, PEG-40 jojoba acid, PEG-40 jojoba alcohol, PEG-40 lanolin, PEG-40 olive glycerides, PEG-40 ricinoleamide, PEG-40 sorbitan diisostearate, PEG-40 sorbitan lanolate, PEG-40 sorbitan laurate, PEG-40 sorbitan perisostearate, PEG-40 sorbitan peroleate, PEG-40 sorbitan stearate, PEG-40 sorbitan tetraoleate, PEG-40 soya sterol, PEG-40 stearate, PEG-42 babassu glycerides, PEG-44 castor oil, PEG-45 hydrogenated castor oil, PEG-45 palm kernel glycerides, PEG-45 safflower glycerides, PEG-45 stearate, PEG-5 castor oil, PEG-5 cocamide, PEG-5 cocamine, PEG-5 cocoate, PEG-5 glyceryl sesquioleate, PEG-5 glyceryl stearate, PEG-5 glyceryl triisostearate, PEG-5 hydrogenated castor oil, PEG-5 hydrogenated corn glycerides, PEG-5 hydrogenated lanolin, PEG-5 hydrogenated tallow amine, PEG-5 lanolate, PEG-5 lanolin, PEG-5 lanolinamide, PEG-5 lauramide, PEG-5 octanoate, PEG-5 oleamide, PEG-5 oleamide dioleate, PEG-5 oleamine, PEG-5 oleate, PEG-5 sorbitan isostearate, PEG-5 soya sterol, PEG-5 soyamine, PEG-5 stearamine, PEG-5 stearate, PEG-5 tallate, PEG-5 tallow amide, PEG-5 tricaprylyl citrate, PEG-5 trimethylolpropane trimyristate, PEG-50 castor oil, PEG-50 hydrogenated castor oil, PEG-50 hydrogenated tallow amine, PEG-50 lanolin, PEG-50 shea butter, PEG-50 sorbitol hexaoleate, PEG-50 stearamine, PEG-50 stearate, PEG-50 tallow amide, PEG-54 castor oil, PEG-54 hydrogenated castor oil, PEG-55 castor oil, PEG-55 hydrogenated castor oil, PEG-55 lanolin, PEG-6 beeswax, PEG-6 caprylic/capric glycerides, PEG-6 cocamide, PEG-6 dilaurate, PEG-6 dioleate, PEG-6 distearate, PEG-6 isolauryl thioether, PEG-6 isopalmitate, PEG-6 isostearate, PEG-6 lauramide, PEG-6 laurate, PEG-6 laurate/tartarate, PEG-6 oleamide, PEG-6 oleate, PEG-6 palmitate, PEG-6 sorbitan beeswax, PEG-6 sorbitan oleate, PEG-6 sorbitan stearate, PEG-6 stearate, PEG-6 undecylenate, PEG-60 almond glycerides, PEG-60 castor oil, PEG-60 corn glycerides, PEG-60 evening primrose glycerides, PEG-60 hydrogenated castor oil, PEG-60 lanolin, PEG-60 sorbitan tetraoleate, PEG-60 sorbitan tetrastearate, PEG-66 trihydroxystearin, PEG-7 cocamide, PEG-7 glyceryl cocoate, PEG-7 hydrogenated castor oil, PEG-7 lanolate, PEG-7 oleamide, PEG-7 oleate, PEG-7 ricinoleate, PEG-7 stearate, PEG-7 tallow amine, PEG-70 hydrogenated lanolin, PEG-70 mango glycerides, PEG-75 castor oil, PEG-75 cocoa butter glycerides, PEG-75 dilaurate, PEG-75 dioleate, PEG-75 distearate, PEG-75 lanolin, PEG-75 lanolin oil, PEG-75 lanolin wax, PEG-75 propylene glycol stearate, PEG-75 shea butter glycerides, PEG-75 shorea butter glycerides, PEG-75 sorbitan lanolate, PEG-78 glyceryl cocoate, PEG-8 beeswax, PEG-8 behenate, PEG-8 C12-18 ester, PEG-8 caprate, PEG-8 caprylate, PEG-8 caprylate/caprate, PEG-8 caprylic/capric glycerides, PEG-8 castor oil, PEG-8 cocoate, PEG-8 di/triricinoleate, PEG-8 dicocoate, PEG-8 diisostearate, PEG-8 dilaurate, PEG-8 dioleate, PEG-8 distearate, PEG-8 ditallate, PEG-8 glyceryl laurate, PEG-8 hydrogenated fish glycerides, PEG-8 hydrogenated tallow amine, PEG-8 isolauryl thioether, PEG-8 isostearate, PEG-8 laurate, PEG-8 myristate, PEG-8 oleate, PEG-8 propylene glycol cocoate, PEG-8 sesquilaurate, PEG-8 sesquioleate, PEG-8 sorbitan beeswax, PEG-8 soyamine, PEG-8 stearate, PEG-8 tallate, PEG-8 tallow amide, PEG-8 undecylenate, PEG-80 glyceryl cocoate, PEG-80 glyceryl tallowate, PEG-80 hydrogenated castor oil, PEG-80 methyl glucose laurate, PEG-80 sorbitan palmitate, PEG-85 lanolin, PEG-9 castor oil, PEG-9 cocoate, PEG-9 diethylmonium chloride, PEG-9 distearate, PEG-9 laurate, PEG-9 oleamide, PEG-9 oleate, PEG-9 stearamide carboxylic acid, PEG-9 stearate, pentaerythrityl distearate, pentaerythrityl tetraisostearate, phosphatidylcholine, polyglyceryl-10 decalinoleate, polyglyceryl-10 decaoleate, polyglyceryl-10 decastearate, polyglyceryl-10 diisostearate, polyglyceryl-10 dioleate, polyglyceryl-10 distearate, polyglyceryl-10 heptaoleate, polyglyceryl-10 heptastearate, polyglyceryl-10 isostearate, polyglyceryl-10 laurate, polyglyceryl-10 mono/dioleate, polyglyceryl-10 myristate, polyglyceryl-10 oleate, polyglyceryl-10 pentaoleate, polyglyceryl-10 pentastearate, polyglyceryl-10 stearate, polyglyceryl-10 tetraoleate, polyglyceryl-10 trioleate, polyglyceryl-10 tristearate, polyglyceryl-2 caprate, polyglyceryl-2 caprylate, polyglyceryl-2 diisostearate, polyglyceryl-2 dioleate, polyglyceryl-2 distearate, polyglyceryl-2 isopalmitate, polyglyceryl-2 isostearate, polyglyceryl-2 lanolin alcohol ether, polyglyceryl-2 laurate, polyglyceryl-2 oleate, polyglyceryl-2 oleyl ether, polyglyceryl-2 sesquiisostearate, polyglyceryl-2 sesquioleate, polyglyceryl-2 sesquistearate, polyglyceryl-2 sorbitan pentacaprylate, polyglyceryl-2 sorbitan tetracaprylate, polyglyceryl-2 stearate, polyglyceryl-2 tetraisostearate, polyglyceryl-2 tetrastearate, polyglyceryl-2 triisostearate, polyglyceryl-2-PEG-4 stearate, polyglyceryl-3 beeswax, polyglyceryl-3 caprate, polyglyceryl-3 cetyl ether, polyglyceryl-3 decyltetradecanol, polyglyceryl-3 dicaprate, polyglyceryl-3 diisostearate, polyglyceryl-3 dioleate, polyglyceryl-3 distearate, polyglyceryl-3 hydroxylauryl ether, polyglyceryl-3 isostearate, polyglyceryl-3 laurate, polyglyceryl-3 methylglucose distearate, polyglyceryl-3 myristate, polyglyceryl-3 oleate, polyglyceryl-3 ricinoleate, polyglyceryl-3 stearate, polyglyceryl-3 stearate SE, polyglyceryl-4 caprate, polyglyceryl-4 cocoate, polyglyceryl-4 isostearate, polyglyceryl-4 laurate, polyglyceryl-4 lauryl ether, polyglyceryl-4 oleate, polyglyceryl-4 oleyl ether, polyglyceryl-4 stearate, polyglyceryl-4-PEG-2 cocamide, polyglyceryl-5 isostearate, polyglyceryl-5 laurate, polyglyceryl-5 oleate, polyglyceryl-6 dioleate, polyglyceryl-6 distearate, polyglyceryl-6 hexaoleate, polyglyceryl-6 isostearate, polyglyceryl-6 laurate, polyglyceryl-6 oleate, polyglyceryl-6 pentaoleate, polyglyceryl-6 pentastearate, polyglyceryl-6 ricinoleate, polyglyceryl-6 tristearate, polyglyceryl-8 oleate, polyglyceryl-8 stearate, PPG-1-PEG-9 lauryl glycol ether, PPG-10 cetyl ether, PPG-10 glyceryl ether, PPG-10 lanolin alcohol ether, PPG-10-buteth-9, PPG-10-ceteareth-20, PPG-12-buteth-12, PPG-12-buteth-16, PPG-12-laneth-50, PPG-12-PEG-50 lanolin, PPG-12-PEG-65 lanolin oil, PPG-15-buteth-20, PPG-15-PEG-11 hydrogenated lauryl alcohol ether, PPG-17-buteth-17, PPG-2 cocamine, PPG-2 isoceteth-20 acetate, PPG-2 lanolin alcohol ether, PPG-2 tallowamine, PPG-2-buteth-3, PPG-2-ceteareth-9, PPG-2-ceteth-10, PPG-2-ceteth-20, PPG-2-deceth-10, PPG-2-isodeceth-12, PPG-2-isodeceth-4, PPG-2-isodeceth-6, PPG-2-isodeceth-9, PPG-2-PEG-6 coconut oil esters, PPG-20 lanolin alcohol ether, PPG-20-buteth-30, PPG-20-decyltetradeceth-10, PPG-20-glycereth-30, PPG-20-PEG-20 hydrogenated lanolin, PPG-23-PEG-4 trimethylolpropane, PPG-24-buteth-27, PPG-24-glycereth-24, PPG-24-PEG-21 tallowaminopropylamine, PPG-25 diethylmonium chloride, PPG-25-laureth-25, PPG-26-buteth-26, PPG-27 glyceryl ether, PPG-28 cetyl ether, PPG-28-buteth-35, PPG-3 hydrogenated castor oil, PPG-3 myristyl ether, PPG-3-buteth-5, PPG-3-isosteareth-9, PPG-3-laureth-9, PPG-3-myreth-11, PPG-3-myreth-3, PPG-30 lanolin alcohol ether, PPG-33-buteth-45, PPG-4 myristyl ether, PPG-4-ceteareth-12, PPG-4-ceteth-1, PPG-4-ceteth-10, PPG-4-ceteth-5, PPG-40 diethylmonium chloride, PPG-40-PEG-60 lanolin oil, PPG-5 lanolate, PPG-5 lanolin alcohol ether, PPG-5 lanolin wax, PPG-5 lanolin wax glyceride, PPG-5 pentaerythrityl ether, PPG-5-buteth-7, PPG-5-ceteth-20, PPG-5-laureth-5, PPG-55 glyceryl ether, PPG-6 C12-18 pareth-11, PPG-6-decyltetradeceth-12, PPG-6-decyltetradeceth-20, PPG-6-decyltetradeceth-30, PPG-66-glycereth-12, PPG-7-buteth-10, PPG-8-ceteth-1, PPG-8-ceteth-10, PPG-8-ceteth-2, PPG-8-ceteth-20, PPG-8-ceteth-5, PPG-9-buteth-12, propylene glycol dicocoate, propylene glycol hydroxystearate, propylene glycol isodeceth-12, propylene glycol isodeceth-4, propylene glycol isostearate, propylene glycol laurate, propylene glycol myristate, propylene glycol oleate, propylene glycol ricinoleate, propylene glycol stearate, ricinoleic acid, ricinoleth-40, Ricinus communis (castor) seed oil, safflower glyceride, sorbeth-3 isostearate, sorbitan caprylate, sorbitan cocoate, sorbitan diisostearate, sorbitan dioleate, sorbitan isostearate, sorbitan laurate, sorbitan oleate, sorbitan palmitate, sorbitan sesquiisostearate, sorbitan sesquioleate, sorbitan sesquistearate, sorbitan stearate, sorbitan triisostearate, sorbitan trioleate, sorbitan tristearate, sorbityl acetate, soy acid, soy sterol, soy sterol acetate, soyaethyl morpholinium ethosulfate, soyamidopropyl dimethylamine, soyamine, soyaminopropylamine, stearamidopropyl dimethylamine stearate, sucrose cocoate, sucrose dilaurate, sucrose distearate, sucrose laurate, sucrose myristate, sucrose oleate, sucrose palmitate, sucrose polylaurate, sucrose polylinoleate, sucrose polyoleate, sucrose polystearate, sucrose ricinoleate, sucrose stearate, sucrose tetrastearate triacetate, sucrose tribehenate, sucrose tristearate, sulfated castor oil, sunflower seed oil glyceride, sunflower seed oil glycerides, synthetic beeswax, or any combination thereof.

In particular examples, the at least one emollient may comprise one or more of glyceryl triacetyl ricinoleate, bis-diglyceryl polyacyladipate-2, hydrogenated polyisobutene, synthetic beeswax, diisostearyl malate, glyceryl distearate, or any combination thereof. In some examples, the at least one emollient may comprise at least glyceryl triacetyl ricinoleate, bis-diglyceryl polyacyladipate-2, hydrogenated polyisobutene, synthetic beeswax, and diisostearyl malate, which may be present in any mass ratio with respect to one another. For example, if present, suitable amounts of glyceryl triacetyl ricinoleate may range from about 10 wt % to about 40 wt %, suitable amounts of bis-diglyceryl polyacyladipate-2 may range from about 5 wt % to about 20 wt %, suitable amounts of hydrogenated polyisobutene may range from about 5 wt % to about 20 wt %, suitable amounts of synthetic beeswax may range from about 5 wt % to about 20 wt %, suitable amounts of diisostearyl malate may range from about 1 wt % to about 15 wt %, and suitable amounts of glyceryl distearate may range from about 0.05 wt % to about 0.8 wt %, each based on total mass of the cosmetic composition.

The cosmetic compositions may optionally further comprise at least one viscosity-decreasing agent. Viscosity-decreasing agents are substances that are capable of altering the viscosity of a composition by making the composition thinner or less viscous. When present, the concentration of the at least one viscosity-decreasing agent may vary over a range of about f1 wt % to about f2 wt %, wherein f1 and f2 are, independently, 0.01, 0.05, 0.1, 0.5, 1, 2, 3, 4, 5, 6, 7, 8, 9, or 10, based on total mass of the cosmetic composition and where f1<f2. For example, the at least one viscosity-decreasing agent may be present in the cosmetic compositions in an amount ranging from about 0.01 wt % to about 10 wt %, or about 0.05 wt % to about 5 wt %, or about 0.5 wt % to about 2 wt %, based on total mass of the cosmetic composition.

Non-limiting examples of viscosity-decreasing agents suitable for use in the cosmetic compositions of the present disclosure may include, but are not limited to, isosorbide derivatives such as isosorbide nitrates, isosorbide esters, isosorbide ethers, and isosorbide phosphates. In some examples, if a viscosity-decreasing agent is present, the viscosity-decreasing agent may comprise at least one isosorbide ether, such as dimethyl isosorbide. For example, suitable amounts of dimethyl isosorbide, if present, may range from about 0.1 wt % to about 2 wt %, based on total mass of the cosmetic composition.

The cosmetic compositions may optionally further comprise one or more particulate materials, such as pigments, inert materials, mixtures thereof, and the like. When present, the concentration of the one or more particulate materials may vary over a range of about g1 wt % to about g2 wt %, wherein g1 and g2 are, independently, 0.01, 0.05, 0.1, 0.5, 1, 2, 3, 4, 5, 6, 7, 8, 9, 10, 15, 20, 25, 30, 35, 40, 45, 50, 55, 60, 65, or 70, based on total mass of the cosmetic composition and where g1<g2. For example, the one or more particulate materials may be present in the cosmetic compositions in an amount ranging from about 0.01 wt % to about 75 wt %, or about 0.05 wt % to about 70 wt %, or about 0.1 wt % to about 65 wt %, or about 0.5 wt % to about 10 wt %, based on total mass of the cosmetic composition.

Suitable particulate materials may be colored or non-colored powders. Any of the powders disclosed herein may be optionally surface treated with lecithin, amino acids, mineral oil, silicone, or various other agents, either alone or in combination, to coat the powder surface and to render the particulates more lipophilic in nature.

Examples of suitable non-colored powders include, for instance, bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or any combination thereof.

Examples of suitable colored powders may include organic or inorganic pigments. Suitable examples of organic pigments may include, for instance, various aromatic types including azo, indigoid, triphenylmethane, anthroquinone, and xanthine dyes which are designated as D&C and FD&C blues, browns, greens, oranges, reds, yellows, and the like. Organic pigments may also include insoluble metallic salts of certified color additives, commonly referred to as the Lakes. Suitable examples of inorganic pigments may include, for instance, iron oxides, ultramarines, chromium, chromium hydroxide colors, or any combination thereof. Iron oxides of red, blue, yellow, brown, black, and mixtures thereof may be suitable for use.

Embodiments disclosed herein include the following:

Embodiment 1. A cosmetic composition comprising:
about 70 wt % to about 85 wt % of at least one emollient, based on total mass; and up to about 0.3 wt % of at least one retinoid, based on total mass.

Embodiment 2. The cosmetic composition of Embodiment 1, wherein the cosmetic composition is a solid.

Embodiment 3. The cosmetic composition of Embodiment 1 or Embodiment 2, wherein the at least one retinoid comprises a retinoid selected from the group consisting of retinol, a retinol ester, retinal, retinoic acid, a retinoic acid ester, isotretinoin, alitretinoin, etretinate, acitretin, adapalene, bexarotene, tazarotene, trifarotene, and any combination thereof.

Embodiment 4. The cosmetic composition of any one of Embodiments 1-3, wherein the at least one retinoid comprises one or more retinoic acid esters.

Embodiment 5. The cosmetic composition of Embodiment 4, wherein the at least one retinoid comprises hydroxypinacolone retinoate.

Embodiment 6. The cosmetic composition of any one of Embodiments 1-5, wherein the at least one emollient comprises an emollient selected from the group consisting of glyceryl triacetyl ricinoleate, bis-diglyceryl polyacyladipate-2, hydrogenated polyisobutene, synthetic beeswax, diisostearyl malate, glyceryl distearate, and any combination thereof.

Embodiment 7. The cosmetic composition of any one of Embodiments 1-6, wherein the at least one emollient comprises at least glyceryl triacetyl ricinoleate.

Embodiment 8. The cosmetic composition of any one of Embodiments 1-7, wherein the at least one emollient comprises at least bis-diglyceryl polyacyladipate-2.

Embodiment 9. The cosmetic composition of any one of Embodiments 1-8, wherein the at least one emollient comprises at least hydrogenated polyisobutene.

Embodiment 10. The cosmetic composition of any one of Embodiments 1-9, wherein the at least one emollient comprises at least synthetic beeswax.

Embodiment 11. The cosmetic composition of any one of Embodiments 1-10, wherein the at least one emollient comprises at least diisostearyl malate.

Embodiment 12. The cosmetic composition of any one of Embodiments 1-11, wherein the at least one retinoid is present in an amount ranging from about 0.0001 wt % to about 0.3 wt %, based on total mass.

Embodiment 13. The cosmetic composition of any one of Embodiments 1-12, further comprising:
about 0.001 wt % to about 5 wt % of at least one humectant, based on total mass.

Embodiment 14. The cosmetic composition of Embodiment 13, wherein the at least one humectant comprises at least caprylyl glycol.

Embodiment 15. The cosmetic composition of any one of Embodiments 1-14, further comprising:
about 5 wt % to about 25 wt % of at least one occlusive, based on total mass.

Embodiment 16. The cosmetic composition of Embodiment 15, wherein the at least one occlusive comprises an occlusive selected from the group consisting of Helianthus annuus (sunflower) seed wax, Oryza sativa (rice) bran wax, squalane, tocopherol, and any combination thereof.

Embodiment 17. The cosmetic composition of any one of Embodiments 1-16, further comprising:
about 0.01 wt % to about 10 wt % of at least one viscosity-decreasing agent, based on total mass.

Embodiment 18. The cosmetic composition of Embodiment 17, wherein the at least one viscosity-decreasing agent comprises at least dimethyl isosorbide.

Embodiment 19. The cosmetic composition of any one of Embodiments 1-18, wherein an amount of the at least one retinoid remains within +/−10% of an initial amount of the at least one retinoid after maintaining the cosmetic composition at 40° C. and 60% relative humidity for about 6 months.

Embodiment 20. A cosmetic composition comprising:
about 70 wt % to about 85 wt % of at least one emollient, based on total mass, the at least one emollient comprising at least glyceryl triacetyl ricinoleate, bis-diglyceryl polyacyladipate-2, hydrogenated polyisobutene, synthetic beeswax, and diisostearyl malate;
about 0.001 wt % to about 0.3 wt % of at least one retinoid, based on total mass, the at least one retinoid comprising at least hydroxypinacolone retinoate;
about 15 wt % to about 25 wt % of at least one occlusive, based on total mass, the at least one occlusive comprising at least Helianthus annuus (sunflower) seed wax, Oryza sativa (rice) bran wax, squalane, and tocopherol;
about 0.5 wt % to about 2 wt % of at least one viscosity-decreasing agent, based on total mass, the at least one viscosity-decreasing agent comprising at least dimethyl isosorbide; and
about 0.005 wt % to about 3 wt % of at least one humectant, based on total mass, the at least one humectant comprising at least caprylyl glycol.

To facilitate a better understanding of the present disclosure, the following examples of preferred or representative embodiments are given. In no way should the following examples be read to limit, or to define, the scope of the invention.

### Examples

Samples were formulated by blending the components listed in Table 1 with heating and then cooling to form a solid composition.

TABLE 1

| | Sample 1 | Sample 2 | Sample 3 |
|---|---|---|---|
| Component | (wt % ) | | |
| hydroxypinacolone retinoate | 0.1 | 0.1 | 0.1 |
| Total Retinoids | 0.1 | 0.1 | 0.1 |
| glyceryl triacetyl ricinolate | 38.4 | 19 | 10.5 |
| bis-diglycerol polyacyladipate-2 | 12 | 15 | 12 |
| hydrogenated polyisobutene | 12 | 15 | 12 |
| synthetic beeswax | 10.5 | 13.5 | 10.2 |
| diisostearyl malate | 9 | 14.8 | 17 |
| glyceryl distearate | 0.3 | 0.4 | — |
| jojoba esters | — | — | 6 |
| C₁₀-C₁₈ triglycerides | — | — | 4 |
| Butyrospermum parkii (shea) butter | — | — | 2 |
| Mangifer indica (mango) seed butter | — | — | 1 |
| Total E mollients | 82.2 | 77.7 | 74.7 |
| squalane | 9.5 | 12 | 9.5 |
| Helianthus annuus | 4 | 5 | 8 |
| (sunflower) seed wax | | | |
| Oryza sativa (rice) bran wax | 3 | 4 | — |
| tocopherol | 0.00048 | 0.0006 | 0.0035ª |
| Total Occlusives | 16.5 | 21.0 | 17.5 |
| dimethyl isosorbide | 0.9 | 0.9 | 0.9 |
| caprylyl glycol | 0.3 | 0.3 | 0.3 |
| silica | — | — | 1.5 |
| mica | — | — | 5 |

^{a}Includes added tocopherol and tocopherol within jojoba esters.

The above samples were tested under two sets of temperature conditions. Each sample was placed in a stability chamber maintained at either room temperature (25° C.±2° C.) or accelerated (elevated) temperature (40° C.±2° C.). Each stability chamber was maintained at a relative humidity of 60%+5%. Stability of the retinoid was monitored at various time points: initially, after one month, after two months, after three months, and after six months. Assessment of the retinoid stability was conducted by removing a sample from the stability chamber and analyzing for hydroxypinacolone retinoate by UPLC/UV-VIS (Waters ACQUITY UPLC H-Class PLUS System with a photodiode array (PDA) detector). A wavelength of 360 nm was used for analyses. The measured concentrations were determined with a measurement accuracy of about +10%. Measured concentrations of hydroxypinacolone retinoate in wt % at selected sampling intervals are shown in Table 2 below. The initial and subsequent concentrations are believed to deviate from the expected 0.1 wt % due to some initial degradation of the stock hydroxypinacolone retinoate.

TABLE 2

| | Sample 1 | | Sample 2 | | Sample 3 | |
|---|---|---|---|---|---|---|
| | (wt %) | | (wt %) | | (wt %) | |
| Time Point | 25° C. | 40° C. | 25° C. | 40° C. | 25° C. | 40° C. |
| Initial | 0.087 | 0.087 | 0.091 | 0.091 | 0.091 | 0.091 |
| 1 month | 0.090 | 0.088 | 0.089 | 0.090 | 0.091 | 0.092 |
| 3 months | 0.094 | 0.090 | 0.092 | 0.092 | 0.098 | 0.095 |
| 6 months | 0.090 | 0.085 | 0.091 | 0.085 | 0.095 | 0.086 |

After 3 and 6 months of stability testing, there were no significant changes in the visual appearance of the samples compared to their initial visual appearances. No changes in color, hue vibrancy, acridity, surface blistering, blemishes, or the like were evident. Further, there were no differences in the visual appearances of the samples aged at the different testing temperatures. As shown in Table 2, the hydroxypinacolone retinoate concentration remained essentially constant within measurement accuracy over the 6-month testing period.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element, or group of elements is preceded with the transitional phrase “comprising,” it is understood that we also contemplate the same composition or group of elements with transitional phrases “consisting essentially of,” “consisting of,” “selected from the group consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is the following:

1. A cosmetic composition comprising:
about 70 wt % to about 85 wt % of at least one emollient, based on total mass; and
up to about 0.3 wt % of at least one retinoid, based on total mass.

2. The cosmetic composition of claim 1, wherein the cosmetic composition is a solid.

3. The cosmetic composition of claim 1, wherein the at least one retinoid comprises a retinoid selected from the group consisting of retinol, a retinol ester, retinal, retinoic acid, a retinoic acid ester, isotretinoin, alitretinoin, etretinate, acitretin, adapalene, bexarotene, tazarotene, trifarotene, and any combination thereof.

4. The cosmetic composition of claim 1, wherein the at least one retinoid comprises one or more retinoic acid esters.

5. The cosmetic composition of claim 1, wherein the at least one retinoid comprises hydroxypinacolone retinoate.

6. The cosmetic composition of claim 1, wherein the at least one emollient comprises an emollient selected from the group consisting of glyceryl triacetyl ricinoleate, bis-diglyceryl polyacyladipate-2, hydrogenated polyisobutene, synthetic beeswax, diisostearyl malate, glyceryl distearate, and any combination thereof.

7. The cosmetic composition of claim 1, wherein the at least one emollient comprises at least glyceryl triacetyl ricinoleate.

8. The cosmetic composition of claim 1, wherein the at least one emollient comprises at least bis-diglyceryl polyacyladipate-2.

9. The cosmetic composition of claim 1, wherein the at least one emollient comprises at least hydrogenated polyisobutene.

10. The cosmetic composition of claim 1, wherein the at least one emollient comprises at least synthetic beeswax.

11. The cosmetic composition of claim 1, wherein the at least one emollient comprises at least diisostearyl malate.

12. The cosmetic composition of claim 1, wherein the at least one retinoid is present in an amount ranging from about 0.0001 wt % to about 0.3 wt %, based on total mass.

13. The cosmetic composition of claim 1, further comprising:
about 0.001 wt % to about 5 wt % of at least one humectant, based on total mass.

14. The cosmetic composition of claim 13, wherein the at least one humectant comprises at least caprylyl glycol.

15. The cosmetic composition of claim 1, further comprising:
about 5 wt % to about 25 wt % of at least one occlusive, based on total mass.

16. The cosmetic composition of claim 15, wherein the at least one occlusive comprises an occlusive selected from the group consisting of Helianthus annuus (sunflower) seed wax, Oryza sativa (rice) bran wax, squalane, tocopherol, and any combination thereof.

17. The cosmetic composition of claim 1, further comprising:
about 0.01 wt % to about 10 wt % of at least one viscosity-decreasing agent, based on total mass.

18. The cosmetic composition of claim 17, wherein the at least one viscosity-decreasing agent comprises at least dimethyl isosorbide.

19. The cosmetic composition of claim 1, wherein an amount of the at least one retinoid remains within +/−10% of an initial amount of the at least one retinoid after maintaining the cosmetic composition at 40° C. and 60% relative humidity for about 6 months.

20. A cosmetic composition comprising:
about 70 wt % to about 85 wt % of at least one emollient, based on total mass, the at least one emollient comprising at least glyceryl triacetyl ricinoleate, bis-diglyceryl polyacyladipate-2, hydrogenated polyisobutene, synthetic beeswax, and diisostearyl malate;
about 0.001 wt % to about 0.3 wt % of at least one retinoid, based on total mass, the at least one retinoid comprising at least hydroxypinacolone retinoate;
about 15 wt % to about 25 wt % of at least one occlusive, based on total mass, the at least one occlusive comprising at least Helianthus annuus (sunflower) seed wax, Oryza sativa (rice) bran wax, squalane, and tocopherol;
about 0.5 wt % to about 2 wt % of at least one viscosity-decreasing agent, based on total mass, the at least one viscosity-decreasing agent comprising at least dimethyl isosorbide; and
about 0.005 wt % to about 3 wt % of at least one humectant, based on total mass, the at least one humectant comprising at least caprylyl glycol.
